(19) United States
(12) Patent Application Publication
(10) Pub. No.: US 2012/0276518 A1
(43) Pub. Date: Nov. 1, 2012

(54) Method and Device for Perfusing Tissue by ExVivo Attachment to a Living Organism

(76) Inventor: John Archie Gillis, Halifax (CA)

(21) Appl. No.: 13/436,977

(22) Filed: Apr. 1, 2012

Related U.S. Application Data
(60) Provisional application No. 61/479,341, filed on Apr. 26, 2011.

(51) Int. Cl. A01N 1/02; C12M 3/00

(52) U.S. Cl. 435/1.1; 435/284.1

(57) ABSTRACT

The present invention is a holding vessel that has bioreactor and perfusion bioreactor components, a temperature specific environment and holes for transporting substances from a living organism.

When the holding vessel is in use it will contain a tissue selection that will be attached to the circulatory system of a living organism by connecting existing vasculature of the organism to engineered or grafted umbilical/vascular cables and then connecting the other end of the umbilical cables to the vasculature of the tissue selection. A tubular construct containing a protective solution will protect the vascular cables.

The tissue selections used will be selected from existing or fabricated tissues, but preference is given to cryogenically prepared tissues electronically dispensed from a three-dimensional printing device.

### CROSS-REFERENCE TO RELATED APPLICATIONS

U.S. Provisional Application No. 61/479,341

### FEDERALLY SPONSORED RESEARCH

Not Applicable

### SEQUENCE LISTING OR PROGRAM

Not Applicable

### BACKGROUND OF THE INVENTION

1. Field of Invention

The present invention relates to methods of perfusing tissue constructs.

2. Prior Art

One of the major challenges facing tissue engineering today is the requirement for more complex functionality. For a greater number of tissue engineered structures to be considered useful in areas such as transplantation, more biomechanical stability is required along with an advanced means of supplying these structures with nutrients and removal of waste products, especially when discussing thick tissue structures.

Bioreactors and perfused bio-reactors have had some success with delivering some of the required nutrients to a construct or existing tissue selection, but designing or discovering better systems for nutrient delivery for tissue constructs or selections is still a major concern.

A major dilemma with most current tissue engineering technologies is that most tissue engineered structures and organs require a means of providing vascularization and perfusion to survive. Creating this vascular supply and more viable methods of perfusion to a thick-engineered tissue construct remains one of the great challenges in the field today.

Tissue engineering was originally considered a sub-field of biomaterials. It has recently grown in both importance and potential and is now considered to be a field of its own. It generally uses a combination of cells, engineering, materials methods, and suitable biochemical and physio-chemical factors to improve or replace biological functions. Tissue engineering is usually describes as an interdisciplinary field incorporating elements of engineering, material and life sciences.

Most recently tissue engineering has begun to incorporate elements of computer aided design and rapid prototyping. The names currently most in use are bioprinting and organ printing.

Vasculature has been bioprinted in the labs of Anthony Atala. It has also been successfully attached to a perfused bioreactor. It is also common in the art to graft vasculature from one location to another location in a patient, or from one patient to another. Xenografts are tissues used from another species. These methods have their place in medical procedures, but immunosuppressant drugs are usually always required when introducing foreign tissues and if the tissue selection contains tissues that are not a good match rejection can occur.

A group from South Carolina as well as a group led by Gabor Forgacs' has recently demonstrated that building a branching intraorgan vascular tree is a realistic and achievable goal. This issue was also addressed by Peter Wu (University of Oregon, USA) who presented applications of LAB in fabricating branch/stem structures with human endothelial cells and T Boland who presented results on thermal inkjet printing of biomaterials and cells for capillary constructs. (Cui X and Boland T 2009 Human microvasculature fabrication using thermal inkjet printing technology Biomaterials 30 6221-7)

Current methods of perfusing a tissue structure are limited, due to time constraints. This is seen in cases of organ donation. When a donated organ is matched with a recipient, it is imperative that the organ reaches the recipient in as short of time as possible. Even with our advanced technologies, helicopters and database matching systems organs are often lost due to injuries during brain death, ischemia, cell death and other causes.

Currently there are a number of systems that are perfusing organs such as Transmedics, “Organ Care System”, Organ Recovery Systems “LifePort” technologies and the Toronto XVIVO Lung Perfusion System. This is a system being worked on by Dr. Shaf Keshavjee in the Lung Transplant Program at Toronto General Hospital (TGH). They have developed an “ex vivo” or outside the body technique capable of continuously perfusing or pumping a bloodless solution containing oxygen, proteins and nutrients into injured donor lungs. This technique allows the surgeons the opportunity to assess and treat injured donor lungs, while they are outside the body, to make them suitable for transplantation.

These methods of perfusion are great advances in medical technologies, but still have their limitations. This is because they are artificial. It seems very unlikely that these and other systems could provide the same biochemical and biomechanical signals, nutrient supply, gas exchange and waste removal system that an actual organism can provide.

In placental mammals, the umbilical cord (also called the birth cord or funiculus umbilicalis) is the connecting cord from the developing embryo or fetus to the placenta. During prenatal development, the umbilical cord comes from the same zygote as the fetus and (in humans) normally contains two arteries (the umbilical arteries) and one vein (the umbilical vein), buried within Wharton's jelly. The umbilical vein supplies the fetus with oxygenated, nutrient-rich blood from the placenta. Conversely, the umbilical arteries return the deoxygenated, nutrient-depleted blood. The umbilical cable is often saved after birth for its cord blood and other uses, but has never been used for perfusing a tissue selection ex vivo.

Tissues are often fabricated in the laboratory using stem cells, growth and differentiation factors, biomaterials, printing devices and biomimetic environments. It is with these combinations of engineered extracellular matrices (or scaffolds), cells, and biologically active molecules that researchers in this field have propelled this area of research forward.

One of the main methods of preserving tissues prior to implantation is through the use of cryoprotectant solutions. A cryoprotectant is a substance that is used to protect biological tissue from freezing damage. This damage often occurs due to the formation of ice. Cryoprotectants in common use include glycols, such as ethylene glycol, propylene glycol and glycerol and dimethyl sulfoxide (DMSO), 2-methyl-2,4-pentanediol (MDP) Sucrose and Trehalose. Cryobiologists have been using both glycerol and dimethyl sulfoxide for decades to reduce ice formation in sperm and embryos that are cold-preserved in liquid nitrogen.

Mixtures of cryoprotectants have less toxicity and are more effective than single-agent cryoprotectants. A mixture of formamide with DMSO, propylene glycol and a colloid was for many years the most effective of all artificially created cryoprotectants. Cryoprotectant mixtures have been used for vitrification, i.e. solidification without any crystal ice formation. Vitrification has important application in preserving embryos, biological tissues and organs for transplant. Vitrification is also used in cryonics in an effort to eliminate freezing damage.

Some cryoprotectants function by lowering a solution's or a material's glass transition temperature. In this way the cryprotectants prevent actual freezing, and the solution maintains some flexibility in a glassy phase.

Vitrification techniques utilize low toxicity solutions and optimized cooling and warming curves that, when applied under sterile conditions, allow for better, longer, safer and more convenient storage of complex living systems.

An example of a method of cryopreservation of tissues by vitrification is Khirabadi; Bijan S., Song; Ying C., Brockbank; Kelvin G. M. “Method of cryopreservation of tissues by vitrification”, Organ Recovery Systems, Inc. U.S. Pat. No. 7,157,222, (2007) or U.S. Pat. No. 6,740,484

This prior art teaches a method that includes vascularized tissues and avascular tissues, or organs. The method comprises immersing the tissue or organ in increasing concentrations of cryoprotectant to a cryoprotectant concentration sufficient for vitrification; rapidly cooling the tissue or organ to a temperature between −80.degree. C. and the glass transition temperature (T.sub.g); and further cooling the tissue or organ from a temperature above the glass transition temperature to a temperature below the glass transition temperature to vitrify the tissue or organ.

This prior art also describes a method for removing a tissue or organ from vitrification in a cryoprotectant solution. The method comprises slowly warning a vitrified tissue or organ in the cryoprotectant solution to a temperature between −80.degree. C. and the glass transition temperature; rapidly warming the tissue or organ in the cryoprotectant solution to a temperature above −75.degree. C.; and reducing the concentration of the cryoprotectant by immersing the tissue or organ in decreasing concentrations of cryoprotectant.

With this method for treating tissues or organs, viability is retained at a high level. For example, for blood vessels, the invention provides that smooth muscle functions and graft patency rate are maintained.

These and similar methods are great for protecting certain portions of existing tissues for a limited amount, but are not often successful at penetrating deep into thicker tissue constructs. It is an object of the present invention to prepare a tissue construct with both intracellular and extracellular cryoprotectant solutions by including the protective solutions during a tissue fabrication process known in the art as bioprinting. The cellular compositions that are to make up the tissue construct will be prepared for preservation prior to or during a bio printing process, thus allowing precise placement of protective solutions, thus when the bioprinting process is completed a tissue construct with the capabilities to be better preserved for a longer duration of time and greater functionality will have been achieved.

Cryoprotectants have rarely if ever been used in tissue engineering. Most cryoprotectants have been used for protecting existing structures. It can be very difficult to position the protective solutions deep within these already existing structures. Lab grown tissue engineered structures are also limited by these same problems. Preserving the tissue selection or construct after it has been fabricated makes it extremely difficult to reach all the desired areas. In the present invention it is the ability of the protective solutions to be selectively located anywhere within the structure that is one of the key benefits of the present invention.

Preservation of organs and tissues are commonplace in medicine, but again because organs are most often donated rather that fabricated it can be difficult to place these solutions in areas that can deeply penetrate the structure, especially if the tissue or organ is a thick structure.

Organ printing is usually assisted by computers, dispenser-based, and has an emphasis on three-dimensional fabrication. These methods are aimed at constructing functional organ modules however at present there has been limited success and the printing of entire organs layer-by-layer has not yet been realized.

Bio-printing or organ printing is a new area of research and engineering that involves printing devices that deposit biological material. Examples of bioprinter technologies would be those in development by Organovo and fabricated at Inventech, which use combinations of “bio-ink” and “bio-paper” to print complex 3D structures.

A number of developments have been occurring in the field of organ printing. One such development is that of Self-Assembling Cell Aggregates. Forgacs; Gabor; (Columbia, Mo.); Jakab; Karoly; (Columbia, Mo.); Neagu; Adrian; (Columbia, Mo.); Mironov; Vladimir; (Mount Pleasant, S.C.) “Self-Assembling Cell Aggregates and Methods of Making Engineered Tissue Using the Same”, The Curators of the Univeristy of Missouri, Columbia Mo., US20080070304, 2008

This prior art describes a composition comprising a plurality of cell aggregates for use in the production of engineered organotypic tissue by organ printing. In a method of organ printing, a plurality of cell aggregates are embedded in a polymeric or gel matrix and allowed to fuse to form a desired three-dimensional tissue structure. An intermediate product comprises at least one layer of matrix and a plurality of cell aggregates embedded therein in a predetermined pattern. Modeling methods predict the structural evolution of fusing cell aggregates for combinations of cell type, matrix, and embedding patterns to enable selection of organ printing processes parameters for use in producing an engineered tissue having a desired three-dimensional structure.

Another development is the method of forming an array of viable cells developed by James Yoo, Tao Xu and Anthony Atala which decribes a method wherein at least two different types of viable mammalian cells are printed on to a substrate. Inventors: James Yoo, Tao Xu, Anthony Atala. Application Ser. No. 12/293,490 Publication number: US 2009/0208466 A1 Filing date: Apr. 20, 2007

These methods of tissue engineering still suffer from some of the limitations of traditional scaffolding methods. There have been some great successes with these methods, but the issue of nutrient delivery is still a major concern.

A common problem with thick tissue structures is that cells deep inside the structure are damaged due to a lack of nutrient delivery. One can delay this problem for a short by preserving the tissue with a cryoprotectant solution, but unless the tissue is prepared as described in the present invention the problems of getting cryoprotectant solutions into all the desired locations, including cells deep within the structure remains a large and limiting problem.

If tissue engineering is ever to surpass the tissue thickness limit of 100-200 μm, it must overcome the challenge of creating functional blood vessels to supply cells with oxygen and nutrients and to remove waste products.

### SUMMARY

The present invention describes a holding vessel that has bioreactor and perfusion bioreactor components, a temperature specific environment and organic vasculature for transporting substances from and to a living organism.

When the holding vessel is in use it will contain a tissue selection that will be attached to the circulatory system of a living organism by connecting the existing vasculature of the organism to engineered or grafted vascular cables. The other ends of the vascular cables are then connected to the vasculature of the tissue selection. A tubular construct containing a protective solution will protect the vascular cables.

The holding vessel will provide support, oxygen and nutrient delivery to the tissue selection. The present methods will provide a novel and superior means of supplying a tissue selection with nutrient delivery along with biochemical and mechanical signals that are superior to known methods.

The tissue selections used will be selected from existing or fabricated tissues, but preference is given to cryogenically prepared tissues electronically dispensed from a three-dimensional printing device.

### DRAWINGS—REFERENCE NUMERALS

10—Protective tube that holds umbilical cord
12—Protective holding vessel for tissue selection(s)
14—Organism with circulatory system that will supply nutrient delivery and waste removal for a tissue selection.
20—Vascular cable, which may house one or more vascular structures
22—Vascular cable inside holding vessel
24—Holding vessel
26—Human arm
28—First layer of breast tissue with newly growing vasculature
30—Second layer of breast tissue with newly growing vasculature
32—Third layer of breast tissue with newly growing vasculature
34—Forth layer of breast tissue with newly growing vasculature
36—Fifth layer of breast tissue with newly growing vasculature
38—Sixth layer of breast tissue with newly growing vasculature
40—Seventh layer of breast tissue with newly growing vasculature
42—Grown Vasculature
44—Mouse
46—Kidney
48—Cryoprotectant Solution
50—One or more cells
52—Preparation of cells for preservation
54—Bio-paper
56—Cryo-prepared cells assembled into self-assembling tissue spheroids/bio-ink
58—Other materials
60—Dispensing system
62—Output from dispensing system, containing spheroid shaped cryo-prepared cellular compositions situated for the process of self-assembly
64—Tissue Construct #1
66—Tissue Construct #2
68—Tissue Construct #3
70—Vat
72—Means of cooling a tissue selection
74—Means of storage and transport
76—Means of warming a tissue selection
78—Means of transferring a tissue selection into a molding system
80—Section or layer of a tissue selection to be assembled into a larger structure.
82—Large tissue structure fabricated from smaller portions

### DETAILED DESCRIPTION OF THE DRAWINGS

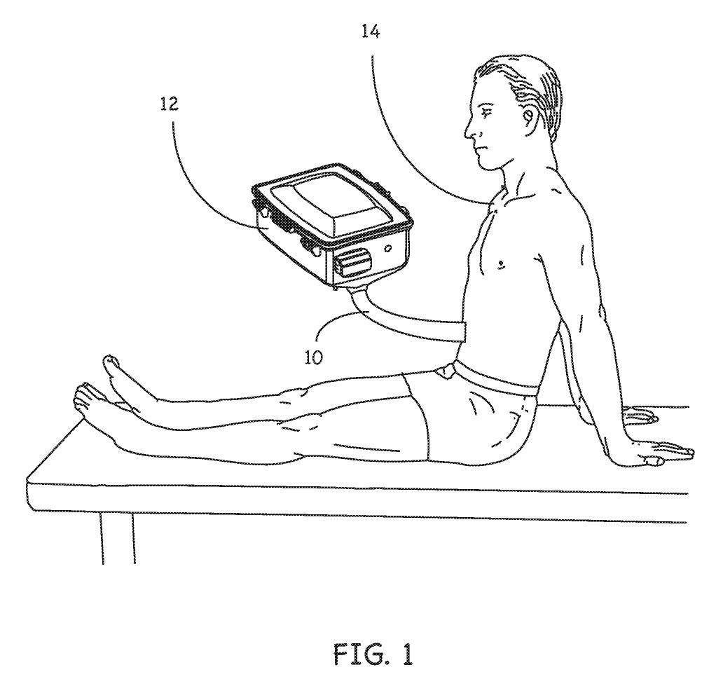

FIG. 1 shows a human being or patient 14 perfusing a tissue structure or organ by means of attachment to their circulatory system. The holding vessel includes a Transmedic device 12 with the organ enclosed inside and is then attached to the human via organic vasculature enclosed in a protective tube 10.

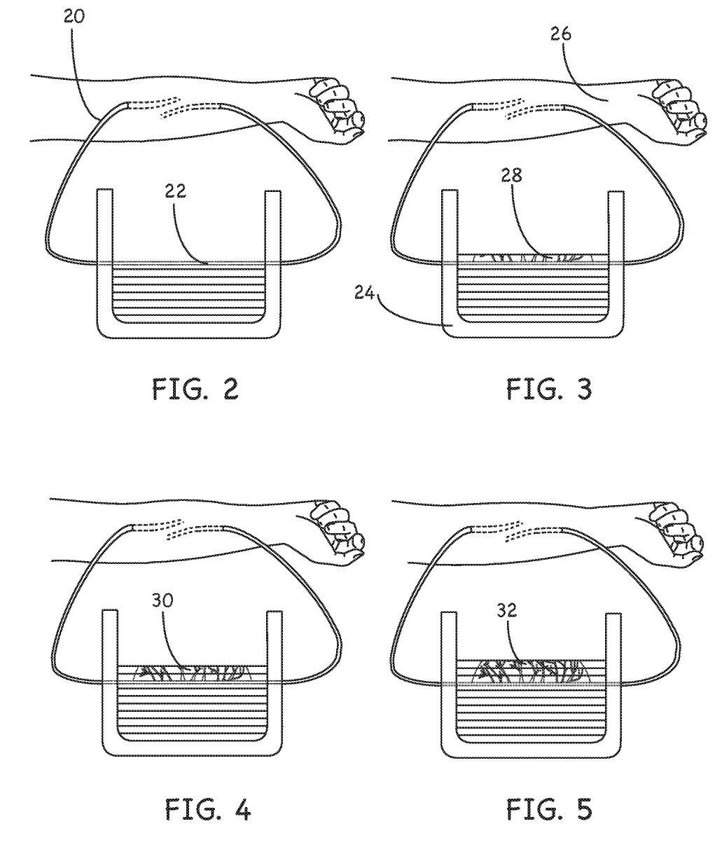

FIG. 2 shows a vascular cable attached to a human arm. Vasculature (and in some instances lymph vessels) are surgically positioned to run through a holding vessel and back to the human arm. The cable first attaches to an artery of the patient and then delivers supplies of blood, oxygen, nutrients, chemical and mechanical signals to the tissue selections located inside the holding vessel. The cable leaving the holding vessel attaches to the veins, which remove waste from the tissue selection. The protective tubing for protecting the structure (that may also include skin, synthetic skin and protective solutions), is not included in this figure.

FIG. 3 shows a layer of breast tissue 28 that was engineered or bioprinted in a thin layer. The layer is printed with extracellular matrix materials and a variety of differentiated cells and is placed in proximity to the vascular cable in our holding vessel. Biological signals known as angiogenic growth factors then activate receptors present on endothelial cells present in the vascular cable attached to the human arm. Activated endothelial cells begin to release enzymes called proteases that degrade the basement membrane to allow endothelial cells to escape from our original (parent) vessel walls. The endothelial cells then proliferate into the surrounding tissue and matrix to form solid sprouts connecting neighboring vessels. As sprouts extend toward the source of the angiogenic stimulus, endothelial cells migrate in tandem, using adhesion molecules, the equivalent of cellular grappling hooks, called integrins. These sprouts then form loops to become a full-fledged vessel lumen as cells migrate to the site of angiogenesis. Sprouting occurs at a rate of several millimeters per day, and enables new vessels to grow across gaps in the vasculature.

FIG. 4 shows a second layer 30 of breast tissue that was engineered or bioprinted in a thin layer. Growth factors, nutrients and other supplies may be added to the holding vessel during the procedure to assist in tissue growth, differentiation, oxygen and nutrient delivery etc. The holding vessel will also be capable of simulating temperature specific environments, such as a human's average temperature of 37 Degrees Celsius.

FIG. 5 shows a third layer 32 of breast tissue that was engineered or bioprinted in a thin layer.

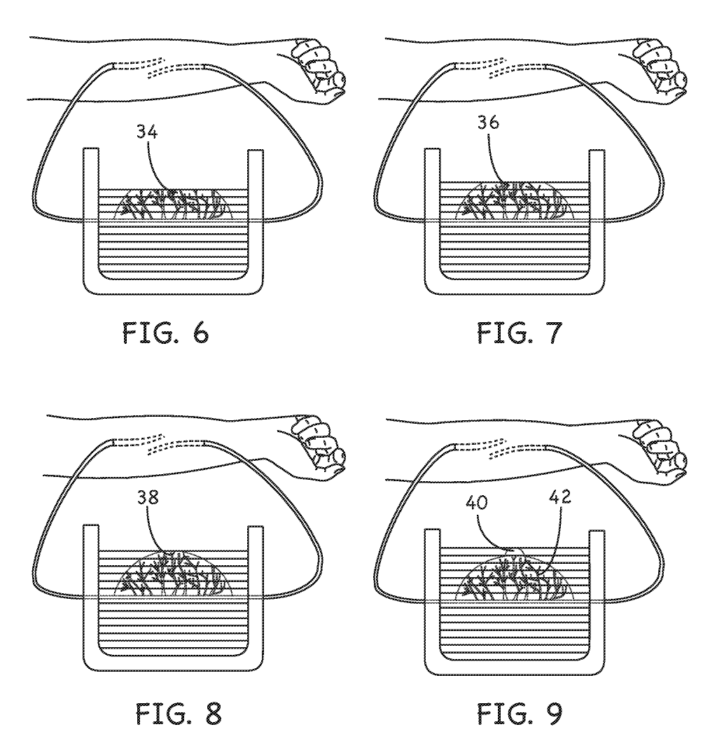

FIG. 6 shows a fourth layer 34 of breast tissue that was engineered or bioprinted in a thin layer.

FIG. 7 shows a fifth layer 36 of breast tissue that was engineered or bioprinted in a thin layer.

FIG. 8 shows a sixth layer 38 of breast tissue that was engineered or bioprinted in a thin layer.

FIG. 9 shows a seventh layer 40 of breast tissue that was engineered or bioprinted in a thin layer.

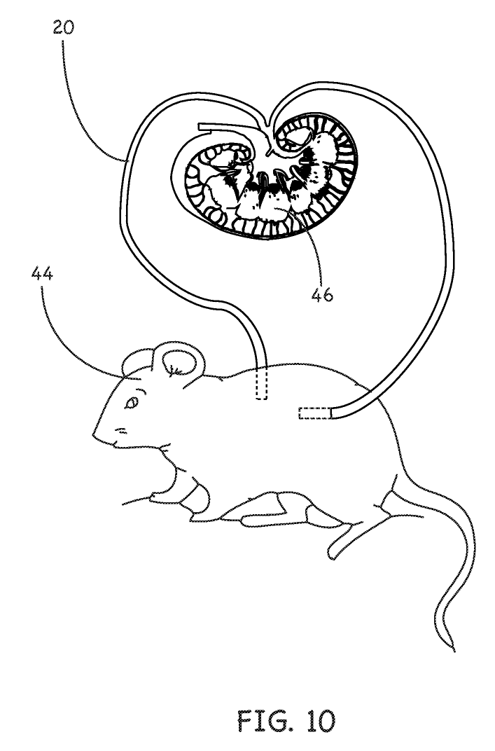

FIG. 10 shows a mouse 44 functioning as a living organic bioreactor. The vascular cable 20 attaches to and perfuses an existing kidney 46.

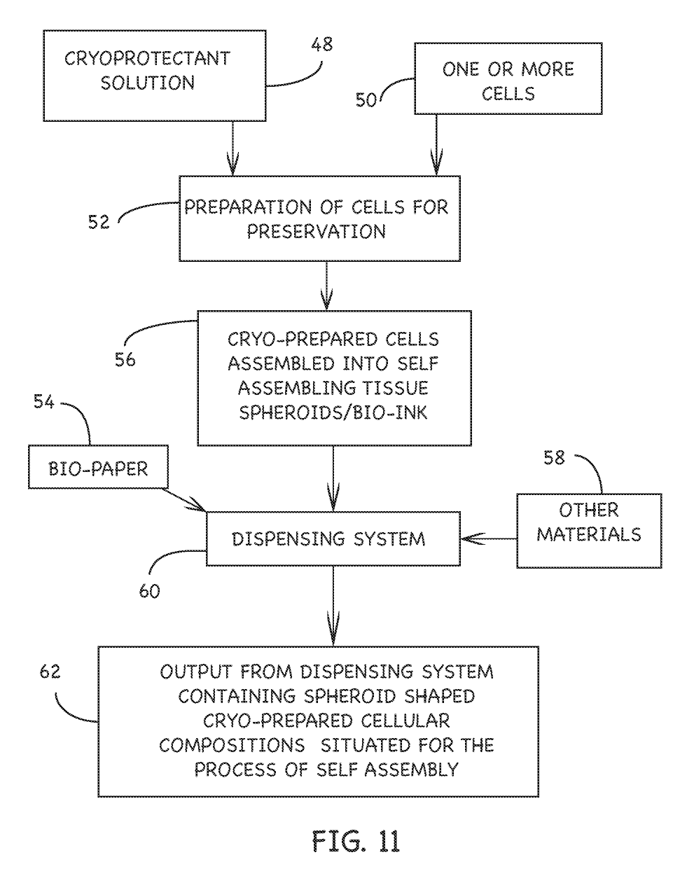

FIG. 11 is a flow chart showing cryoprotectant solutions 48 and one or more cells 50 coming together wherein they are provided with a means of being prepared for preservation 52. The prepared cells of 52 are assembled into self-assembling tissue spheroids or what is known in the art as bio-ink 56. Loaded into a dispensing system 60 are the bio-ink 56, the bio-paper 16 and other materials 20 which may include other cryoprotectant solution, matrix materials, scaffolds and gels. From the dispensing system 60 we get an output-containing spheroid shaped cryo-prepared cellular compositions situated for the process of self-assembly 62 into a desired shape, pattern or three-dimensional structure.

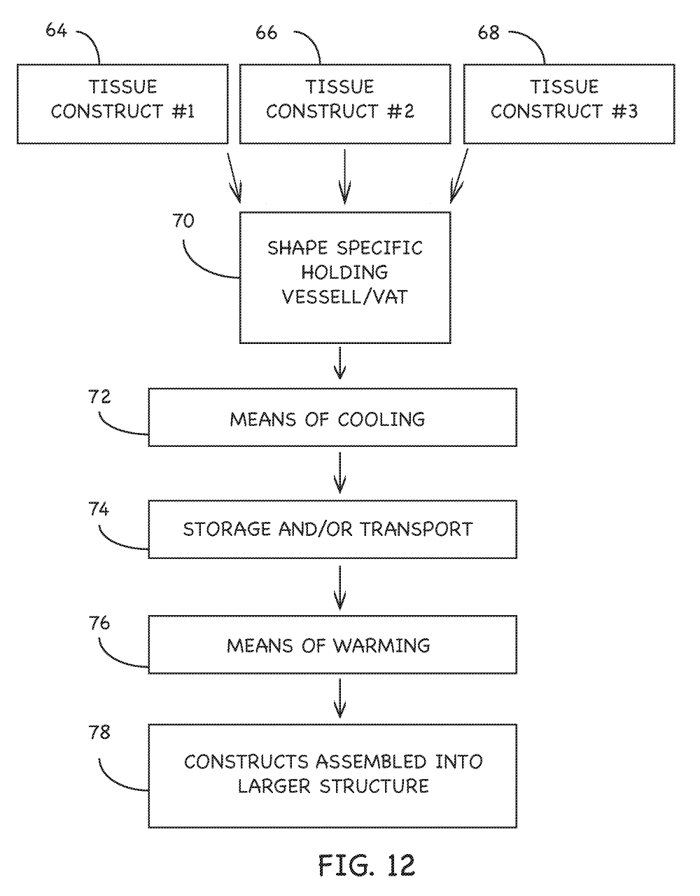

FIG. 12 is a flow chart showing a number of different tissues 64, 66, 68 that will be loaded into a vat 70 with a shape complementary to the shape of the printed tissue selections. A means of cooling 72 will be provided and when cooled to a desired temperature the tissues will be stored and/or transported 74. When the tissues reach their location or it is desired to remove them from their cryopreserved state a means of warming 76 will be provided so as to enable transfer to a pin molding system 78 or for other uses.

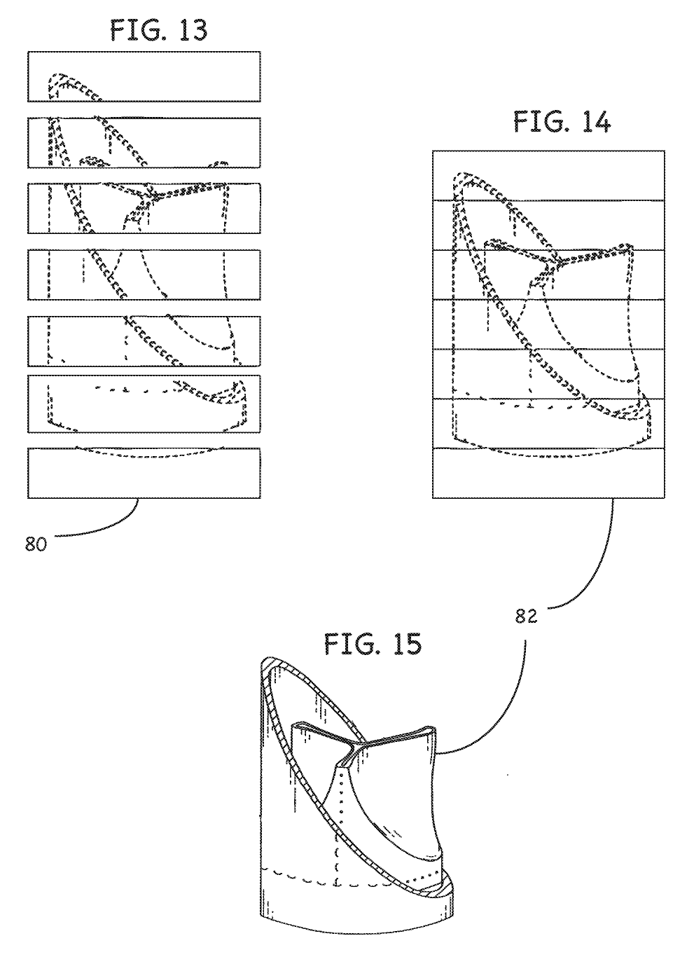

FIG. 13 is a diagram of a heart valve printed in layers or sections 80. Each section 80 was printed as a separate unit with a specific shape. At this stage of the process we can see how when the layers are placed together that they will form the shape of a heart valve.

FIG. 14 is a diagram of our layers 80 stacked together to form a structure 82 that will be coaxed into self-assembly and form the shape of a heart valve.

FIG. 15 shows what separate sections of a heart valve could look like once fully assembled into a finished structure 82.

### DETAILED DESCRIPTION OF PREFERRED EMBODIMENTS

Reference will now be made in detail to various embodiments of the invention, one or more examples of which are set forth below. Each embodiment is provided by way of explanation of the invention, not limitation of the invention. In fact, it will be apparent to those skilled in the art that various modifications and variations may be made in the present invention without departing from the scope or spirit of the invention. For instance, features illustrated or described as part of one embodiment, may be used in another embodiment to yield a still further embodiment. Thus, it is intended that the present invention cover such modifications and variations as come within the scope of the appended claims and their equivalents.

In the preferred embodiments the present invention describes a novel method for the perfusion and vascularization of a tissue selection. In the preferred embodiment the selection consists of tissue-engineered constructs, but may also be very useful in perfusing naturally occurring structures. The invention also consists of a novel holding vessel. This holding vessel may contain standard bioreactor or perfused bioreactor components that include temperature specific programming capabilities, but its novelty lies in the fact that some of its components are actually made from real biological tissues. This creates an environment with the capacity to truly protect and maintain a tissue selection ex vivo, while a living organism is perfusing it. The holding vessel also has attachable tubes for connecting the vascular structures to and from the living organism.

In certain embodiments a layer of real or synthetic skin protects the vascular structures, which are then located in the protective tubular structures themselves. The protective tubing provides a safe environment for the vasculature in the new unnatural exvivo environment and also prevents the patient from viewing the site of attachment and the organic materials used in the attachment, as this could potentially be an unsettling experience.

To create an engineered structure that is capable of being perfused is a great challenge, and perfusion of naturally existing structures is currently achieved using perfused bioreactors. It is doubtful that they will at any time in the near future achieve tissue survival success rates anywhere close to a natural organic environment.

By using a living organism as our bioreactor we get to utilize all the biochemical signaling factors and waste removal systems that are already in place and perfectly developed. To attach an existing organ to the present invention is quite simple, as it just requires surgical attachment of vascular cables to and from the organ to be perfused and also to the organism that is functioning as the bioreactor.

The more complicated procedure of developing a tissue-engineered structure that can be perfused is novel to the present invention and is described as follows. The entire process begins by immersing cultured cells that are aggregated into self-assembling tissue spheroids in varying levels of cryoprotectant solutions.

The innovative method comprises ink jet printing a cell composition onto a substrate wherein the cells within the composition have been prepared for cryopreservation, cooling, freezing or vitrification. A great example of Ink jet printing of viable cells is U.S. Pat. No. 7,051,654 Boland; Thomas (Suwanee, Ga.), Wilson, Jr.; William Crisp (Easley, S.C.), Xu; Tao (Clemson, S.C.), which is hereby incorporated by reference in its entirety. It describes a method for forming an array of viable cells. In one embodiment, the method comprises ink-jet printing a cellular composition containing cells onto a substrate. Upon printing, at least about 25% of the cells remain viable after incubation for 24 hours at 37.degree. C. in a 5% CO.sub.2/95% O.sub.2 environment.

In the preferred embodiment the cultured cells that are included in the cellular composition to be printed are prepared with varying levels of cryoprotectant solutions. A variety of solutions can be used to generate various levels of results and successes. Examples of some potential methods that may be used in whole or in part include, but are not limited to “Method of cryopreservation of tissues by vitrification” (Khirabadi; Bijan S., Song; Ying C., Brockbank; Kelvin G. M. “Method of cryopreservation of tissues by vitrification”, Organ Recovery Systems, Inc. U.S. Pat. No. 7,157,222, 2007),

The cryogenically prepared cells will form bio ink that will be loaded into a three-dimensional fabrication device. A great example of a bio ink is US Patent Application 20080070304 to Forgacs; Gabor; (Columbia, Mo.); Jakab; Karoly; (Columbia, Mo.); Neagu; Adrian; (Columbia, Mo.); Mironov; Vladimir; (Mount Pleasant, S.C.) “Self-Assembling Cell Aggregates and Methods of Making Engineered Tissue Using the Same”, which is hereby incorporated by reference in its entirety and explains bio ink and bio paper.

No prior art reference provides a description of a process incorporating the use of cryogenic preparation of cells or cell aggregates for the purpose of being loaded into a printer. This is one of the novel features of the present invention. With prior methods of applying cryoprotectant solutions to some tissue constructs, (especially into thick constructs or organs) it has been found difficult if not impossible to get the cryoprotectant solutions to the desired locations. The present invention provides a remedy for this problem.

After being dispensed from an ink jet printer the cellular spheroids or aggregates will be preserved by methods of freezing or vitrification. The construct at this point in time can be stored and transported for cell therapies or drug testing, but in the preferred embodiment of the present invention it is used as a section to be fused with other similar sections to create a larger construct. Once taken out of their preserved state they will be coaxed into self-assembly and fused together to create a larger structure.

The cryogenically prepared cells will be printed in layers, and as the layers are completed they are put into a vitrified or frozen state. The layers are organized so that when they are ready, they will fit together in a desired shape or pattern that will allow the proper portions to fuse in the correct areas.

In the preferred embodiment self-assembly may occur after preservation, however in alternative embodiments it will occur prior to preservation.

When the layered structures are taken out of their vitrified state they will be coaxed into self-assembly as is described in US Patent Application 20080070304, unless they were coaxed into self-assembly prior to preservation. It is an object of the present invention to provide a means for fusing these tissue layers into a larger more elaborate vascularized and perfused structure.

The present sectioning method can be used without the use of cryogenic solutions integrated into the construction process, but the tissue layers would need to be made available in a very timely manner at selected intervals, which could be more difficult to achieve without preservation or immediate delivery to the holding vessel of the present invention.

The holding vessel of the present invention may contain elements used in bioreactors, perfused bioreactors or systems for ex vivo care at near physiologic conditions. It will also have holes for attaching one ore more vascular cables, that will deliver substances such as blood, nutrients, gases and growth factors both to and away from the tissue selection that will be held within. Vasculature has been bioprinted in the labs of Anthony Atala without cryopreservation included to a limited degree. It has also been successfully attached to a perfused bioreactor.

The first tissue selection to be delivered to the holding vessel will be the vascular cables themselves. The vascular or umbilical cables may be fabricated from human cells, donated from an existing organism or may be donated by a suitably matched newborn baby. The cables are then attached to a human circulatory system, which would likely be the circulatory system of the future recipient of the structure. The cables are run through our holding vessel and then back to the human circulatory system.

The vascular cables in our holding vessel will be directly accessible by the engineering professional creating the product. The vascular structures held within the vessel may not necessarily have a layer of skin, artificial skin or tubular structures protecting them. It will be the components of standard bioreactors, perfused bioreactors or systems for exvivo care at near physiologic conditions with temperature specific programming components that will protect the structures held within.

A first layer of external tissue will be prepared for placement into the holding vessel and if necessary it will be warmed to a selected temperature, such as 37 degrees Celsius. It will then be placed directly on the vascular structure located within the holding vessel. Growth factors that promote angiogenesis will be added to the tissue selections and in a short period of time the structures begin to fuse or self assemble. Venules and capillaries will form that will provide a means of vascularization for the structure along with perfusion for maintaining further growth, aggregation and blood vessel development. This is similar to the processes that occur in fetal development and cancerous tumor growths.

A second layer of external tissue will be prepared for placement into the holding vessel and again if necessary it will be warmed to a selected temperature, such as 37 degrees Celsius. It will then be placed in proximity to the existing vascularized/vascularizing structure located within the holding vessel. Growth factors that promote angiogenesis will again be added to the newer tissue selections and in a short period of time the new structure will begin to fuse or self assemble with the first structures placed in the holding vessel. Venules and capillaries will form that will provide a means of vascularization for the new larger structure. The initial vascular cables will provide the structure with means of perfusion for maintaining further growth, aggregation, blood vessel development and also waste removal.

A third, fourth and fifth layers of external tissue will be prepared for placement into the holding vessel in a similar fashion and the process will continue until the structure is considered to be completed. Numerous layers of skin tissue may also be attached. Once the structure is completed the structure can be removed and implanted into a patient.

One of the great benefits of the structure being located outside of the body is that it may be tended to by doctors, engineers and other professionals for other additional procedures, tests or substance delivery that may be beneficial to the survival and maintenance of the structure. The present methods also make it very easy for the structures to receive external electrical stimuli, which could be of great interest when working with cardiac tissues. Other great benefits of the structure being perfused by the patient's own circulatory system, yet essentially being located outside the body is that it can be much more easily accessed, repaired, manipulated and supplied with additional substances or therapies than are currently available with other methods.

The present invention describes what at first seems odd, but is actually the most natural method of perfusing either a transplanted organ or a tissue engineered construct. If we think of how a fetus is perfused in the womb we have a fetus attached to an umbilical cord, which is attached to its mother via a placenta. Both the fetus and the umbilical are in a protective solution. In the present invention we create something very similar. Our fetus is our tissue engineered construct and our mother is the person who will be having the construct or organ implanted into them.

In the preferred embodiment the exvivo perfusion module will be attached via existing or fabricated umbilical cables to the construct or organ to be perfused. The construct or organ will be located outside of the body and housed in a protective temperature specific environment, likely at 37 degrees C. and may include a protective solution for surrounding the construct/organ. The tube attaching to the recipients circulatory system via an umbilical cable will be housed in a tube containing a protective solution, which may contain Wharton's Jelly or a suitable substitute, nutrient composition, or liquid that may assist in sustaining the cord during perfusion of the construct. Connection of this cord will require surgical attachment.

In some embodiments of the present invention immunosuppressant drugs may need to be administered to the organism functioning as the bioreactor. If the vascular cables are allografts, (taken from a genetically non-identical donor of the same species) or xenograft, it is very likely that immunosuppressant drugs to prevent rejection will be required. One of the great benefits of the present invention however is that once the tissue selection being perfused is completed, or is ready to be implanted and removed from its state of attachment to the living organism, the organism, which in many instances will also be the patient, can be taken off of the immunosuppressant drugs, because all of the foreign tissues will be removed from contact with the patient. In this example it is just the vasculature that connects the tissue engineered construct to the organism functioning as a bioreactor that is an allograft. The engineered structure to be implanted is an autograft.

In yet another embodiments of the present invention the organism functioning, as a bioreactor will simply have their vasculature removed from an internal position (in vivo) to an external position (ex vivo). A surgical procedure will extract vasculature from the organism and position it such that it is located outside the body. Once located outside the body, it will be placed into a protective environment and used as our perfused bioreactor. In this embodiment our holding vessel will attach itself around this existing vasculature.

Successful perfusion of an extra organ using a similar procedure in vivo has been accomplished in the art by what is known as heterotopic surgery. In this medical procedure the patient's own heart is not removed before implanting a donor heart. The donor heart is positioned so that the chambers and blood vessels of both hearts can be connected to form what is effectively a ‘double heart’.

Another example of in vivo perfusion of an extra organ is that of a kidney transplant. In many kidney transplants the original but likely damaged kidneys are left in the recipient.

An example of ex vivo perfusion is that of babies who are occasionally born with organs outside their body and often survive this way for many months prior to having the organs transferred inside their body.

Langer and Vacanti were able to perfuse a tissue construct by inserting scaffold materials seeded with cells into the body of a mouse, under the skin. They were able to mimic the environment in which cells naturally grow and thus were able to unlock the biochemical signals that influence growth and development.

The present invention differs from these procedures because the heterotopic procedure takes place inside an organism not via an ex vivo attachment. Another difference is that in the present invention the living organism that the construct or organ is first attached to after being created acts as a temporary lobby area. Once the organ has been matured in it's temporary location it will be implanted into the recipient.

The tissue constructs of the present invention include portions of, or whole tissues (i.e., bone, cartilage, blood vessels, bladder, etc.) The tissue harvested may consist of any biological material and may include materials that have been manipulated and/or changed from their original state, such as geneticially altered materials or stem cell cultivations.

Current methods of perfusing a tissue structure are limited, due to time constraints. This is seen in cases of organ donation. When a donated organ is matched with a recipient, it is imperative that the organ reaches the recipient in as short of time as possible. Even with our advanced technologies, helicopters and database matching systems organs are often lost, due to a variety of reasons that include injuries during brain death, ischemia, cell death and other causes.

Currently there are a number of systems that are perfusing organs at near physiologic conditions such as Transmedics, “Organ Care System”, Organ Recovery Systems “LifePort” technologies and the Toronto XVIVO Lung Perfusion System. This is a system being worked on by Dr. Shaf Keshavjee in the Lung Transplant Program at Toronto General Hospital (TGH). They have developed an “ex vivo” or outside the body technique capable of continuously perfusing or pumping a bloodless solution containing oxygen, proteins and nutrients into injured donor lungs. This technique allows the surgeons the opportunity to assess and treat injured donor lungs, while they are outside the body, to make them suitable for transplantation.

These methods of perfusion are great advances in medical technologies, but still have their limitations. The present invention describes what at first seems odd, but is actually the most natural method of perfusing either a transplanted organ or a tissue engineered construct. If we think of how a fetus is perfused in the womb we have a fetus attached to an umbilical cord, which is attached to its mother. Both the fetus and the umbilical are in a protective solution. In the present invention we create something very similar. Our fetus is our tissue engineered construct and our mother is the person who will be having the construct or organ implanted into them.

In the preferred embodiment the ex vivo perfusion module will be attached via existing or fabricated umbilical cables to the construct or organ to be perfused. The construct or organ will be located outside of the body and housed in a protective temperature specific environment, likely at 37 degrees C. and may include a protective solution for surrounding the construct/organ. The tube attaching to the recipients circulatory system via an umbilical cable will be housed in a tube containing a protective solution, which may contain Wharton's Jelly or a suitable substitute, nutrient composition, or liquid that may assist in sustaining the cord during perfusion of the construct. Connection of this cord will require surgical attachment.

In placental mammals, the umbilical cord (also called the birth cord or funiculus umbilicalis) is the connecting cord from the developing embryo or fetus to the placenta. During prenatal development, the umbilical cord comes from the same zygote as the fetus and (in humans) normally contains two arteries (the umbilical arteries) and one vein (the umbilical vein), buried within Wharton's jelly. The umbilical vein supplies the fetus with oxygenated, nutrient-rich blood from the placenta. Conversely, the umbilical arteries return the deoxygenated, nutrient-depleted blood.

The computer aided design, manufacturing, assembly and/or printing system of the present invention includes design, manufacturing, assembly and/or printing system that make use of computer technology to aid in the design, manufacturing, assembly and/or printing of a product. Examples of such systems include, Direct Digital Manufacturing, Rapid Prototyping, Three Dimensional Printing, Bio-printing, (CAD/CAM), Stereolithography, Solid Freeform Fabrication, Self-Replicating Machines, 3D Microfabrication, Digital Fabrication and Desktop Manufacturing Systems, and the methods and technologies involved, developed and understood by those skilled in the art.

The Bio-printing systems of the present invention will include the use of what is known in the art as bio-paper and bio-ink.

### Alternative Embodiments

In one alternative embodiment the described perfusion methods can be used to perfuse re-cellularized organs, such as those fabricated by Doris Taylor and other researchers in such patent applications as application Ser. No. 12/064,613, publication number: US 2009/0202977 A1, with filing date: Aug. 28, 2006. This invention provides for methods and materials to decellularize an organ or tissue as well as methods and materials to recellularize a decellularized organ or tissue.

In another alternative embodiment the present invention's ex vivo human perfusion methods could assist with donor organ care. As an example if patient A lives in California and needs a kidney and patient B lives in Boston and needs a kidney, we could have the following scenario. Donor organs become available, but Organ #1 in California is a poor match for Patient A and Organ #2 in Boston is a poor match for Patient B. Patient A has a family member or friend that is willing to perfuse the kidney while traveling to Boston. Patient B has a family member or friend that is willing to perfuse the kidney while traveling to California. Both patients receive kidneys that may have otherwise gone to waste, been damaged due to ischemia poor preservation or any other number of reasons.

It does seem like a lot to ask of a friend or family member, but it seems like a more practical scenario than asking a living friend or family member to go into surgery and give up one of their kidneys forever, which is a relatively frequent procedure.

In another alternative embodiment the present invention will utilize genetically altered animals for assistance with the maturation of tissue constructs or for perfusing tissue selections or organs.

When organs are transplanted between species, immune attack is swift and severe. Pigs for example and other animals have a specific sugar not present in humans and old-world primates. So when a pig organ is transplanted into a baboon, for example, antibodies circulating in the baboon's blood immediately swarm and attack the pig tissue, leading to the death of the organ.

As one example, scientists (particularly David Sachs, the director of the Transplantation Biology Research Center at MGH) made a major advance in overcoming this immune barrier in 2002 by creating genetically engineered pigs that lack the enzyme that attaches the sugar to the surface of pig cells. In a paper published in Nature Medicine, Sachs showed that baboons given kidneys from these genetically modified pigs lived for up to 83 days, far longer than the average 30-day survival time for animals receiving regular pig kidneys.

The tissue selection is attached to a swine designed to lack an immune system in a surgical process. The tissue selection remains in a system for ex-vivo organ care at near-physiologic conditions, but is also attached to a swine by means of an umbilical cable. This procedure allows for many beneficial outcomes, such as providing a preferred environment for organ repair, maturation, transport and the use of an animal rather than a human for the perfusion of the tissue selection or organ.

In another alternative embodiment the present invention will open the door for researching electrical signal and information transfer from one brain tissue selection to another external tissue selection. We could someday be in a position to create an external hard drive for our brains, similar to that of a computer.

This is quite interesting, but where this type of research will become most fascinating, is when we are able to establish connections from one mind to another, similar to how two or more computers can be networked. When the research community begins to better understand developmental neurobiology, intracellular signaling, neuroimmunology, information theory and the numerous information storage and transfer methods of the central nervous system, we will be ready for some dramatic advances.

We will also need to overcome and better understand factors leading to glial scar formation, which significantly inhibits nerve regeneration. Studies with new methods have confirmed that adult CNS neurons have regenerative capabilities, but studies done by researchers have found that the damaged environments do not support and may actually prevent regeneration.

Sharing of thoughts has been documented in a number of conjoined twins. The fact that they were born this way is what likely keeps them from suffering psychosis that would likely occur if attempted with individuals who did not experience thought sharing from birth. It is assumed that with enough research that these challenges will someday be overcome.

Ethical questions, moral philosophy and the many belief systems we have studied and know will need to be re-evaluated and perhaps re-shaped as individuality could start to be questioned. A realm of collective consciousness could be created and could lead us to new ways for educating ourselves. It may also lead us to new discoveries in observer based reality theories, wave function collapse in quantum mechanics and much, much more.

I claim:

1. A method of providing substance transfer for one or more tissue selections comprising;
a. means for attaching said tissue selections to the circulatory system of a living organism ex vivo
b. means of attaching at least one cord with means capable of delivering substances to said tissue selection from said circulatory system and
c. means of attaching at least one cord with a means capable of removing unwanted substances from said tissue construct and
d. a protective holding vessel for said tissue selection.

2. The method of claim 1 wherein said means of attaching said tissue selection to said circulatory system of a living organism is accomplished by the use of an umbilical cord with means capable of transporting nutrients, blood supplies, growth factors, amino acids, electrolytes, gases, hormones, blood cells and other organic materials.

3. The method of claim 1 wherein said at least one cord is an umbilical cable selected from a group consisting of organic vasculature, engineered vascular tissue structures, and non organic units.

4. The method of claim 3 wherein said cord is immersed in a protective solution and tube.

5. The method of claim 4 wherein said protective tube contains a selection of Wharton's Jelly, nutrients, and other protective substances.

6. The method of claim 1 wherein said tissue selection is a donor organ or engineered structure.

7. The method of claim 1 wherein the living organism is genetically modified.

8. The method of claim 7 wherein the living organism is genetically modified to lack an immune system.

9. The method of claim 1 wherein the one or more tissue selections are placed into said holding vessel in layers and at different times.

10. A holding vessel comprising:
a. means capable of receiving substance delivery from a living organism and delivering said substance to a tissue selection held within said holding vessel and
b. means capable of removing substances from said tissue selection and returning said substances to said organism.

11. The holding vessel according to claim 10 wherein said holding vessel contains bioreactor and perfused bioreactor components and means for creating a temperature specific environment.

12. The holding vessel according to claim 10 wherein the means capable of receiving and delivering substances is made from living organic vasculature.

13. A method of producing a tissue construct prepared for preservation at low temperatures comprising, the dispensing of a cellular composition containing at least one cell with at least one cryoprotectant solution from an electronic dispensing system and means for providing self assembly for one or more cellular compositions to fuse into a larger tissue construct.

14. The method of claim 13 wherein said cellular composition containing at least one cell with said at least one cryoprotectant solution is prepared for cryopreservation prior to dispensing.

15. The method of claim 13 wherein said cellular composition containing at least one cell with said at least one cryoprotectant solution is prepared for cryopreservation after being released from said dispensing system.

16. The method of claim 13 wherein said dispensing system comprises a selection of computer aided design, manufacturing and assembly systems, ink jet printers, bio-printing and organ-printing systems.

17. The method of claim 13 wherein said cellular composition consists of one or more self-assembling tissue spheroids.

18. The method of claim 13 further including said one or more cells being prepared with varying levels of cryoprotectant solutions before placing them into said dispensing system.

19. The method of claim 13 wherein said cryoprotectant solution is any substance that is used to protect biological tissue from freezing damage.

20. The method of claim 13 wherein said dispensing system further includes one or more separate cartridges filled with content selected from the group consisting of different cryogenically prepared cells, cryoprotectant solutions, growth factors, matrix materials, nutrients, hydrogen sulfide, lithium.
